(19) United States
(12) Patent Application Publication
(10) Pub. No.: US 2018/0088103 A1
(43) Pub. Date: Mar. 29, 2018

(54) FLUOROPHORES FOR RECYCLED CONTENT VERIFICATION

(71) Applicant: International Business Machines Corporation, ARMONK, NY (US)

(72) Inventors: SARAH K. CZAPLEWSKI, ROCHESTER, MN (US); JOSEPH KUCZYNSKI, NORTH PORT, FL (US); MELISSA K. MILLER, RALEIGH, NC (US); REBECCA N. MORONES, BERTHOUD, CO (US)

(21) Appl. No.: 15/280,382

(22) Filed: Sep. 29, 2016

(51) Int. Cl. G01N 33/44; G01N 21/64; C08J 3/20; C08J 11/04

(52) U.S. Cl. CPC G01N 33/442; G01N 21/64; C08J 2400/22; C08J 11/04; C08J 2400/30; C08J 3/20

(57) ABSTRACT

A process of utilizing one or more fluorophores to identify recycled content includes receiving, at a compounding entity, recycled material that is collected and sorted by a sorting entity. The process includes adding one or more fluorophores to the recycled material to form a recycled resin. The process also includes forming a mixed material from a resin blend that includes a first weight percentage of the recycled resin and a second weight percentage of virgin resin. The process further includes collecting spectral data for the mixed material and storing the spectral data for the mixed material for subsequent recycled content verification testing.

### BACKGROUND

For recycled content verification testing, analytical testing on virgin versus recycled thermoplastics may be ineffective, as both resins bear identical chemical signatures. Verifiable measurement is critical to programs such as any waste reduction program and green procurement programs as Electronic Product Environmental Assessment tool (EPEAT). The ability to accurately measure recycled content with instrumentation enables “green” claims to be verified.

### SUMMARY

According to an embodiment, a process of utilizing one or more fluorophores to identify recycled content is disclosed. The process includes receiving, at a compounding entity, recycled material that is collected and sorted by a sorting entity. The process includes adding one or more fluorophores to the recycled material to form a recycled resin. The process also includes forming a mixed material from a resin blend that includes a first weight percentage of the recycled resin and a second weight percentage of virgin resin. The process further includes collecting spectral data for the mixed material and storing the spectral data for the mixed material for subsequent recycled content verification testing.

According to another embodiment, a process of verifying recycled content based on spectral is disclosed. The process includes receiving, at a verification entity, a thermoplastic material having a claimed recycled content identified by a compounding entity. The process includes collecting spectral data for the thermoplastic material and comparing the spectral data for the thermoplastic material to spectral data collected by the compounding entity. The process also includes selectively validating the claimed recycled content based on a result of the comparison of the spectral data for the thermoplastic material to the spectral data collected by the compounding entity.

According to another embodiment, a process of forming a thermoplastic material having recycled content that is verifiable based on spectral data is disclosed. The process includes receiving, at a manufacturing entity, a mixed material from a compounding entity. The mixed material includes recycled content and non-recycled content. The process also includes forming a thermoplastic material from the mixed material. The process further includes providing first spectral data associated with the mixed material collected by the compounding entity to a verification entity. The verification entity may verify a weight percentage of the recycled content in the thermoplastic material based on a comparison of second spectral data associated with the thermoplastic material to the first spectral data.

The foregoing and other objects, features and advantages of the invention will be apparent from the following more particular descriptions of exemplary embodiments of the invention as illustrated in the accompanying drawings wherein like reference numbers generally represent like parts of exemplary embodiments of the invention.

### BRIEF DESCRIPTION OF THE DRAWINGS

FIG. 1 is a diagram illustrating a process of utilizing fluorophore(s) for recycled content verification, according to one embodiment.

FIG. 2 is a flow diagram showing a particular embodiment of a process of utilizing fluorophore(s) for recycled content verification.

FIG. 3 is a flow diagram showing a particular embodiment of a process of verifying a claimed recycled content.

### DETAILED DESCRIPTION

In the context of testing to verify a claimed recycled portion of a thermoplastic material, it may be difficult and/or expensive to verify the accuracy of a claimed recycled portion because resin formed from recycled material (also referred to herein as “recycled resin”) and resin formed from virgin material (also referred to herein as “virgin resin”) are chemically identical. The present disclosure describes the utilization of fluorophores (also referred to herein as “fluorescence markers” or “markers”) to distinguish between non-recycled content and recycled content.

In the present disclosure, following collection and sorting, a compounder may add a predetermined quantity of one or more fluorophores to recycled material and may then compound or extrude this master batch with virgin material. The fluorescence intensity of the resulting mixed content pellets is then determined and correlated with a weight percentage of recycled content. In some cases, different fluorophores (or combinations of fluorophores) may be added to identify different sources of recycled content. For example, one set of fluorophores may be used to identify post-industrial recycled content, and another set of fluorophores may be used to identify post-consumer recycled content.

When plastic materials that are formed from the mixed content pellets are selected for verification testing, the fluorescence intensity can be easily determined and traced back to a known mix percentage. For example, a verification entity may collect spectral data for a thermoplastic material with a claimed recycled content. The verification entity may compare the spectral data to spectral data from a compounding entity and/or a manufacturing entity. If the spectral data matches, the verification entity may validate the accuracy of the claimed recycled content.

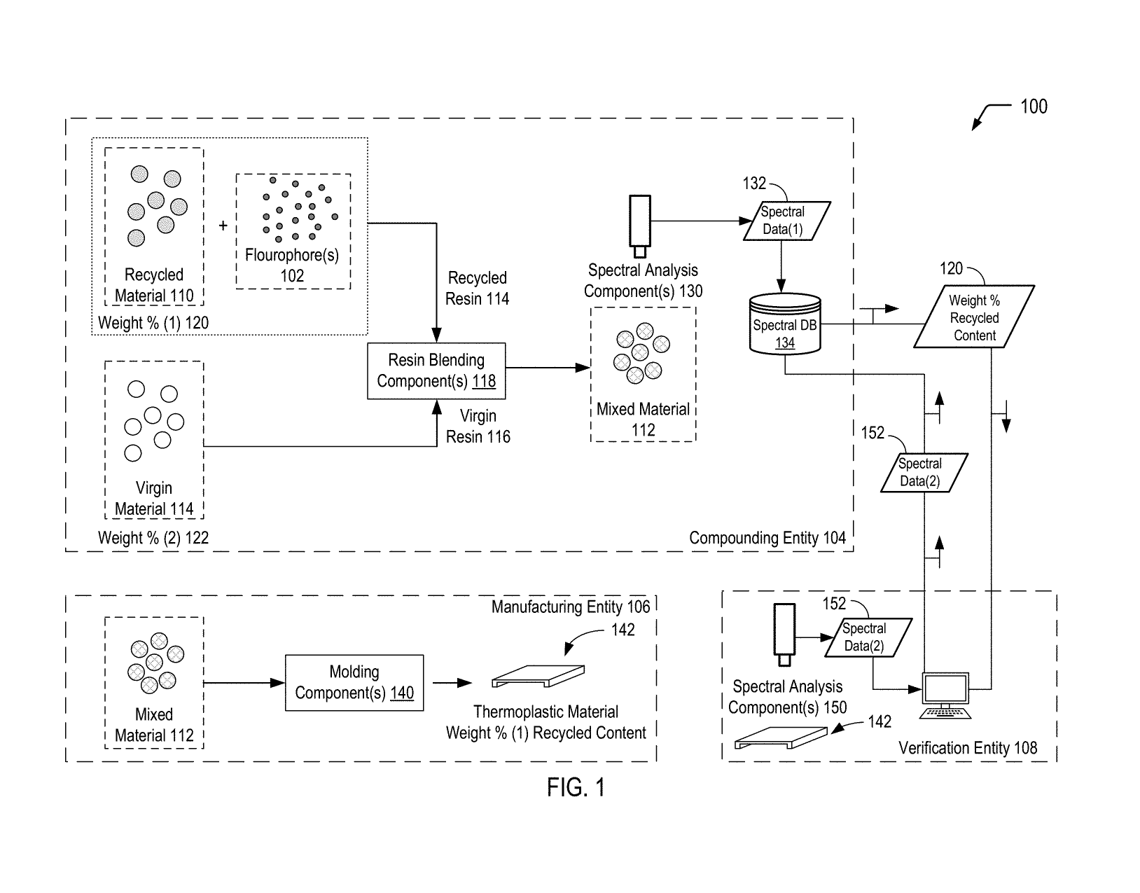

Referring to FIG. 1, a diagram 100 illustrates a particular embodiment of a process of utilizing one or more fluorophores 102 for recycled content verification. In the particular embodiment depicted in FIG. 1, a compounding entity 104, a manufacturing entity 106, and a verification entity 108 are illustrated. The compounding entity 104 receives recycled material 110 that is collected and sorted by a sorting entity (not shown in FIG. 1). The compounding entity 104 adds a known quantity of the fluorophore(s) 102 to the recycled material 110 as a “marker” of recycled content for subsequent comparison to data collected by the verification entity 108 in order to verify the accuracy of a claimed recycled content percentage. It will be appreciated that the example depicted in FIG. 1 is for illustrative purposes only and that alternative and/or additional entities may be perform one or more operations described herein. For example, while the compounding entity 104 and the manufacturing entity 106 are depicted as separate entities in FIG. 1, in other cases, the compounding entity 104 may also manufacture thermoplastic materials that include a blend of recycled content and virgin content.

Following collection and sorting, FIG. 1 illustrates that the compounding entity 104 may add a predetermined quantity of the fluorophore(s) 102 to the recycled material 110. The compounding entity 104 may utilize one or more resin blending components 118 (e.g., an extruder) to compound or extrude recycled resin 114 with virgin resin 116 to form pellets of the mixed material 112. Thermoplastic materials typically comprise one or more polymers. Suitable base resins include, but are not limited to, polyethylene terephthalate (PET or PETE), high-density polyethylene (HDPE), polyethylene chloride (PVC), low-density polyethylene (LDPE), polypropylene (PP), polystyrene (PS), polycarbonate (PC), acrylonitrile butadiene styrene (ABS), and blends thereof.

The recycled resin 114 represents a first weight percentage 120 of the mixed material 112, and the virgin resin 116 represents a second weight percentage 122 of the mixed material 112. While not shown in the example of FIG. 1, in some cases, different fluorophores (or combinations of fluorophores) may be added to identify different sources of recycled content (e.g., post-industrial recycled content and post-consumer recycled content).

In a particular embodiment, the fluorophore(s) 102 may emit fluorescence light somewhere in the wavelength range between approximately 450 nm and 1000 nm. For this region, adequate and cost effective imaging sensors are available and fluorescence from most plastic additives is not significant. Preferably, a loading level of the fluorophore(s) 102 within the recycled resin 114 is low in order to avoid an adverse impact on the chemical and/or physical properties of the recycled resin 114. For example, the loading level of the fluorophore(s) 102 within the recycled resin 114 may be less than 1 weight percent, preferably less than 0.1 weight percent, and more preferably 0.01 weight percent.

As an illustrative, non-limiting example, the fluorophore(s) 102 may include one or more transition metal complexes. In a particular embodiment, tris-chelated 1,2-diimine transition metal ion complexes (especially group VIII: Ru²⁺ and Os²⁺). A non-limiting, illustrative example of transition metal complex includes Ru(bpy)₃²⁺ (where bpy denotes 2,2′-bipyridine). In the case of such luminescent transition metal complexes, light emission is phosphorescence from a spin-forbidden (triplet) excited state. In a particular embodiment, a solid state fluorophore may include Ru(bpy)₃Cl₂ (available from Aldrich Chemical). As another example, the solid state fluorophore may include Ru(bpy)₃(PF₆)₂. Excitation of thermoplastic material containing Ru(bpy)₃²⁺ results in a pronounced fluorescence with an emission peak at about 610 nm.

FIG. 1 illustrates that the mixed material 112 that includes the fluorophore(s) 102 may be subsequently analyzed using one or more spectral analysis components 130 to collect spectral data 132 (identified as “Spectral Data(1)” in FIG. 1) for subsequent recycled content verification testing by the verification entity 108. For example, the spectral analysis component(s) 130 may collect fluorescence intensity data for the mixed material 112 and store the fluorescence intensity data in a spectral database 134 for subsequent use by the verification entity 108 to verify the accuracy of a claimed recycled portion of a thermoplastic material 142 formed from the mixed material 112.

In FIG. 1, after the compounding entity 104 has collected the spectral data 132 for the mixed material 112, the manufacturing entity 106 may utilize one or more molding components 140 to form a thermoplastic material 142 from the mixed material 112. If the thermoplastic material 142 is subsequently selected (e.g., randomly) for verification testing by the verification entity 108, the fluorescence intensity data can be easily determined and traced back to a known mix percentage. For example, the verification entity 108 may utilize one or more spectral analysis components 150 (that may be the same or similar to the one or more spectral analysis components 130 utilized by the compounding entity 104) to collect spectral data 152 (identified as “Spectral Data(2)” in FIG. 1). In the example of FIG. 1, the verification entity 108 may determine whether the spectral data 152 collected for the thermoplastic material 142 matches the spectral data 132 collected by the compounding entity 104 for the mixed material 112 in order to verify that the weight percentage of the recycled material 110 in the thermoplastic material 142 corresponds to the weight percentage 120 that is claimed by the compounding entity 104. When the verification entity 108 determines that the spectral data 152 for the thermoplastic material 142 matches the spectral data 132 from the compounding entity 104, the verification entity 108 may validate that the thermoplastic material 142 includes the claimed portion of recycled content. When the verification entity 108 determines that the spectral data 152 for the thermoplastic material 142 does not match the spectral data 132 from the compounding entity 104, the verification entity 108 may invalidate the claim that the thermoplastic material 142 includes the claimed portion of recycled content.

Thus, FIG. 1 illustrates an example of a process of utilizing one or more fluorophores for recycled content verification. In FIG. 1, a compounding entity adds a known amount of fluorophore(s) to recycled material and collects spectral data for a mixed material including the recycled content and virgin content. The spectral data for the mixed material is subsequently utilized by a verification entity in order to verify the accuracy of a claimed recycled content percentage in a thermoplastic material that is formed from the mixed material. When the spectral data matches, the verification entity may validate the claim that the thermoplastic material includes the claimed recycled percentage. When the spectral data does not match, the verification entity may invalidate the claim that the thermoplastic material includes the claimed recycled percentage.

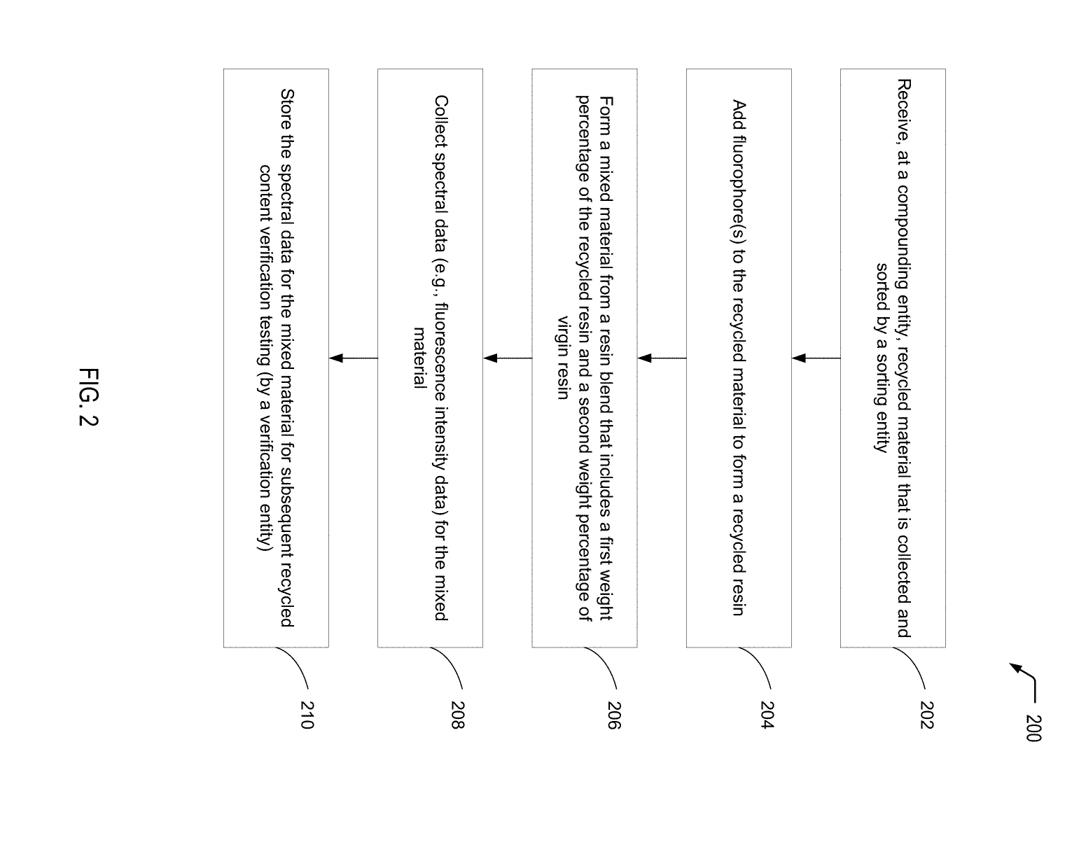

Referring to FIG. 2, a flow diagram depicts an example of a process 200 of utilizing one or more fluorophores for recycled content verification testing. In a particular embodiment, the operations depicted in FIG. 2 may be performed by the compounding entity 104 illustrated in FIG. 1.

The process 200 includes receiving, at a compounding entity, recycled material that is collected and sorted by a sorting entity, at 202. For example, referring to FIG. 1, the compounding entity 104 may receive the recycled material 110 from a sorting entity (not shown in FIG. 1).

The process 200 includes adding one or more fluorophores to the recycled material, at 204. For example, referring to FIG. 1, the compounding entity 104 may add the one or more fluorophores 102 to the recycled material 110. In some cases, the compounding entity 104 may add different fluorophores (or combinations of fluorophores) to the recycled material 110 in order to identify different sources of recycled content (e.g., post-industrial recycled content and post-consumer recycled content).

The process 200 includes forming a mixed material from a resin blend that includes recycled resin and virgin resin, at 206. The recycled resin represents a first weight percentage of the mixed material, and the virgin resin represents a second weight percentage of the mixed material. For example, referring to FIG. 1, the compounding entity 104 may utilize the resin blending component(s) 118 to form the mixed material 112 from a resin blend that includes the recycled resin 114 and the virgin resin 116. The recycled resin 114 may represent the first weight percentage 120 of the resin blend, and the virgin resin 116 may represent the second weight percentage 122 of the resin blend.

The process 200 includes collecting spectral data for the mixed material, at 208. For example, referring to FIG. 1, the compounding entity 104 may utilize the spectral analysis component(s) 130 to collect the spectral data 132 for the mixed material 112. As an example, the spectral data 132 may include fluorescence intensity data associated with the mixed material 112.

The process 200 includes storing the spectral data for the mixed material for subsequent recycled content verification testing, at 210. For example, referring to FIG. 1, the compounding entity 104 may store the spectral data 132 for the mixed material 112 in the spectral database 134. As described further herein, the spectral data 132 for the mixed material 112 may subsequently be utilized by the verification entity 108 to validate the accuracy of a claimed recycled content percentage.

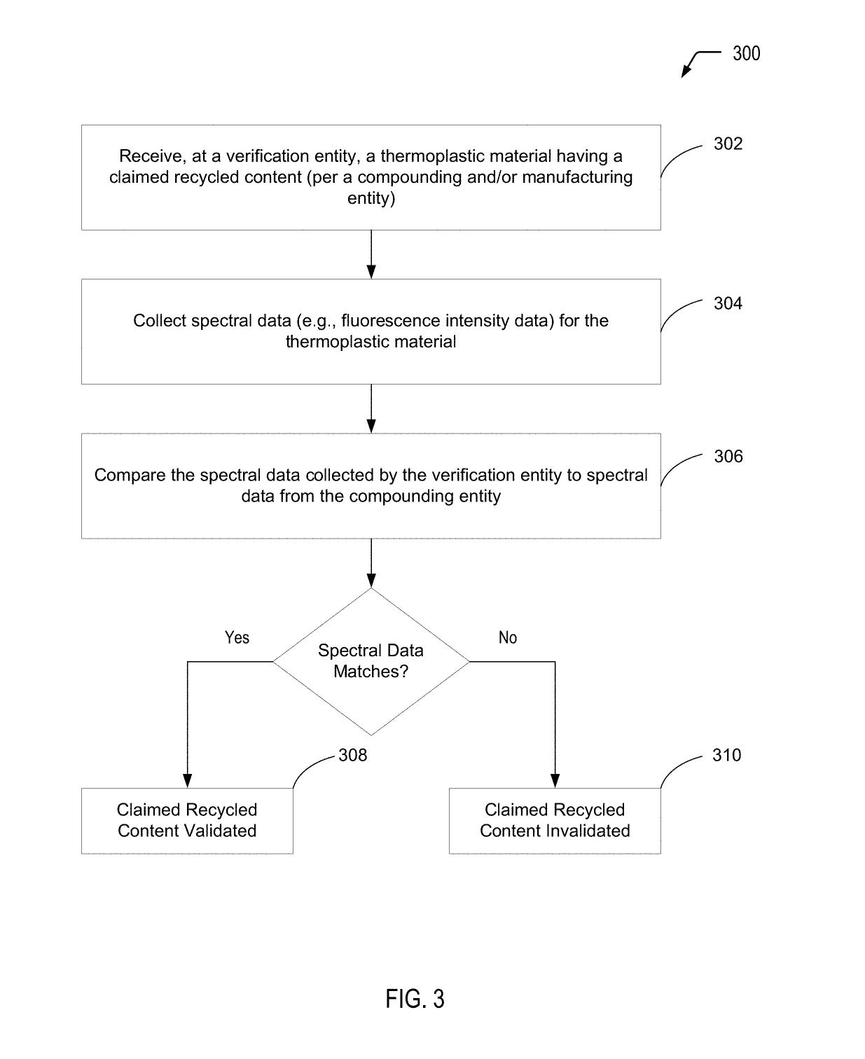

Thus, FIG. 2 illustrates an example of a process of utilizing one or more fluorophores for recycled content verification testing. The compounding entity adds a known amount of fluorophore(s) to recycled material and collects spectral data for a mixed material including the recycled content and virgin content. As illustrated and described further herein with respect to FIG. 3, the spectral data collected by the compounding entity for the mixed material may subsequently be utilized by a verification entity in order to verify the accuracy of a claimed recycled content percentage in a thermoplastic material that is formed from the mixed material.

Referring to FIG. 3, a flow diagram depicts an example of a process 300 of recycled content verification testing. In a particular embodiment, the operations depicted in FIG. 3 may be performed by the verification entity 108 illustrated in FIG. 1.

The process 300 includes receiving, at a verification entity, a thermoplastic material having a claimed recycled content (per a compounding and/or manufacturing entity), at 302. For example, referring to FIG. 1, the verification entity 108 may receive the thermoplastic material 142 that is manufactured by the manufacturing entity 106. The compounding entity 104 may assert that the mixed material 112 includes a particular weight percentage of recycled content, the manufacturing entity 106 may assert that the thermoplastic material 142 includes a particular weight percentage of recycled content, or a combination thereof.

The process 300 includes collecting spectral data for the thermoplastic material, at 304. For example, referring to FIG. 1, the verification entity 108 may utilize the spectral analysis component(s) 150 to collect the spectral data 152 for the thermoplastic material 142. In some cases, the spectral data 152 may include fluorescence intensity data.

The process 300 includes comparing the spectral data collected by the verification entity to spectral data from the compounding entity, at 306. For example, referring to FIG. 1, the spectral data 152 collected by the verification entity 108 for the thermoplastic material 142 may be compared to the spectral data 132 collected by the compounding entity 104 for the mixed material 112. Alternatively, the verification may be performed by the verification entity 108 using pellets of the mixed material 112 (received from either the compounding entity 104 or the manufacturing entity 106).

When the spectral data collected by the verification entity matches the spectral data from the compounding entity, FIG. 3 illustrates that the verification entity may validate the claimed recycled content, at 308. When the spectral data collected by the verification entity does not match the spectral data from the compounding entity, FIG. 3 illustrates that the verification entity may invalidate the claimed recycled content, at 310. For example, referring to FIG. 1, when the spectral data 152 collected by the verification entity 108 matches the spectral data 132 collected by the compounding entity 104, the verification entity 108 may validate that the thermoplastic material 142 includes the claimed weight percentage 120 of recycled content. While not shown in the example of FIG. 1, when the spectral data 152 collected by the verification entity 108 does not match the spectral data 132 collected by the compounding entity 104, the verification entity 108 may invalidate the claim that the thermoplastic material 142 includes the claimed weight percentage 120 of recycled content.

Thus, FIG. 3 illustrates an example of a process of recycled content verification testing. In FIG. 3, spectral data collected by a verification entity may be compared to spectral data collected by a compounding entity. When the spectral data matches, the verification entity may validate a claimed recycled percentage. When the spectral data does not match, the verification entity may invalidate the claimed recycled percentage.

It will be understood from the foregoing description that modifications and changes may be made in various embodiments of the present invention without departing from its true spirit. The descriptions in this specification are for purposes of illustration only and are not to be construed in a limiting sense. The scope of the present invention is limited only by the language of the following claims.

1. A process of utilizing one or more fluorophores to identify recycled content, the process comprising:
receiving, at a compounding entity, recycled material that is collected and sorted by a sorting entity;
adding one or more fluorophores to the recycled material to form a recycled resin;
forming a mixed material from a resin blend that includes a first weight percentage of the recycled resin and a second weight percentage of virgin resin;
collecting spectral data for the mixed material; and
storing the spectral data for the mixed material for subsequent recycled content verification testing by a verification entity.

2. The process of claim 1, wherein the spectral data includes fluorescence intensity data for the mixed material.

3. The process of claim 1, wherein adding the one or more fluorophores to the recycled material includes:
adding a first fluorophore to identify a first source of recycled content; and
adding a second fluorophore to identify a second source of recycled content.

4. The process of claim 3, wherein the first fluorophore is added to the recycled material to identify the first source of recycled content as a post-industrial recycled content source, and wherein the second fluorophore is added to the recycled material to identify the second source of recycled content as a post-consumer recycled content source.

5. (canceled)

6. The process of claim 1, wherein the one or more fluorophores include one or more transition metal ion complexes.

7. The process of claim 6, wherein the one or more transition metal ion complexes include Group VIII transition metal complexes.

8. The process of claim 6, wherein the one or more transition metal ion complexes include Ru(bpy)₃²⁺, wherein bpy denotes 2,2′-bipyridine.

9. The process of claim 1, wherein the one or more fluorophores include one or more solid state fluorophores.

10. A process of verifying recycled content based on spectral data, the process comprising:
receiving, at a verification entity, a thermoplastic material having a claimed recycled content identified by a compounding entity;
collecting spectral data for the thermoplastic material;
comparing the spectral data for the thermoplastic material to spectral data collected by the compounding entity;
selectively validating the claimed recycled content based on a result of the comparison of the spectral data for the thermoplastic material to the spectral data collected by the compounding entity.

11. The process of claim 10, wherein the claimed recycled content is validated when the spectral data for the thermoplastic material matches the spectral data collected by the compounding entity.

12. The process of claim 10, wherein the claimed recycled content is invalidated when the spectral data for the thermoplastic material does not match the spectral data collected by the compounding entity.

13. The process of claim 10, wherein the spectral data collected for the thermoplastic material includes fluorescence intensity data.

14. The process of claim 13, wherein the fluorescence intensity data includes data associated with fluorescence light emitted from the thermoplastic material having a wavelength in a range of 450 nm to 1000 nm.

15. The process of claim 14, wherein the fluorescent light emitted by the thermoplastic material has an emission peak at about 610 nm.

16. A process of forming a thermoplastic material having recycled content that is verifiable based on spectral data, the process comprising:
receiving, at a manufacturing entity, a mixed material from a compounding entity, the mixed material including recycled content and non-recycled content;
forming a thermoplastic material from the mixed material; and
providing first spectral data associated with the mixed material collected by the compounding entity to a verification entity, the verification entity to verify a weight percentage of the recycled content in the thermoplastic material based on a comparison of second spectral data associated with the thermoplastic material to the first spectral data.

17. The process of claim 16, wherein the mixed material includes one or more transition metal ion complexes.

18. The process of claim 17, wherein the one or more transition metal ion complexes include Group VIII transition metal ion complexes.

19. The process of claim 17, wherein the one or more transition metal ion complexes include Ru(bpy)₃²⁺, wherein bpy denotes 2,2′-bipyridine.

20. The process of claim 16, wherein the first spectral data includes fluorescence intensity data associated with fluorescence light emitted from the mixed material having a wavelength in a range of 450 nm to 1000 nm.
